(19) United States
(12) Patent Application Publication
(10) Pub. No.: US 2022/0160954 A1
(43) Pub. Date: May 26, 2022

(54) INFUSION HEAD WITH CONTROLLED RELEASE OF SECONDARY DRUG

(71) Applicant: Eli Lilly and Company, Indianapolis, IN (US)

(72) Inventors: Mary Teresa CARTER, Boston, MA (US); Daniel Morris HARTMANN, Arlington, MA (US); Kristine Alina Bunker MOORE, Cambridge, MA (US); Monica Rixman SWINNEY, Stoneham, MA (US)

(21) Appl. No.: 17/602,383

(22) PCT Filed: Apr. 15, 2020
(86) PCT No.: PCT/US2020/028195

§ 371 Date: Oct. 8, 2021

Related U.S. Application Data
(60) Provisional application No. 62/834,678, filed on Apr. 16, 2019.

(51) Int. Cl. A61M 5/14; A61M 5/142; A61M 5/168

(52) U.S. Cl. CPC A61M 5/1407; A61M 5/14248; A61M 2005/14252; A61M 2202/0007; A61M 5/168

(57) ABSTRACT

Drug infusion systems are disclosed including an infusion head configured to deliver a primary drug and a secondary drug to a patient's subcutaneous tissue. The secondary drug may be provided in a controlled-release material and/or a drug-containing reservoir. Methods for assembling and using such drug infusion systems are also disclosed. The secondary drug may extend the wear time of the drug infusion systems.

### CROSS REFERENCE TO RELATED APPLICATION

This application claims priority to U.S. Provisional Patent Application Ser. No. 62/834,678, filed Apr. 16, 2019, the disclosure of which is hereby expressly incorporated by reference herein in its entirety.

### FIELD OF THE DISCLOSURE

The present disclosure relates to drug infusion systems, more specifically drug infusion systems configured to deliver a primary drug and a secondary drug to a patient's subcutaneous tissue, and to methods for assembling and using the same.

### BACKGROUND OF THE DISCLOSURE

Drug infusion systems are provided for delivering a drug (e.g., insulin) into a patient's subcutaneous tissue. Such drug infusion systems may include a drug delivery device (e.g., pump), an intermediate tubing set, and an infusion head having an infusion element in the form of a cannula or a needle that extends into the patient's skin. In use, the drug may be delivered from the drug delivery device, through the tubing set, through the infusion head and into the patient's subcutaneous tissue via the cannula or the needle.

The patient's body may exhibit an inflammatory and/or foreign body response to the drug infusion system, particularly at the site of the cannula or the needle. For this reason, known drug infusion systems are currently indicated for two- to three-day use. After even a short wear time, the inflammatory and/or foreign body response may impair the efficacy of the patient's infusion site, thereby limiting insulin uptake, increasing the risk of hyperglycemia, and limiting viable infusion site longevity. The limited wear time for the drug infusion system represents a two- to seven-times discrepancy compared with the wear time for continuous glucose monitors (CGMs), thus introducing an obstacle to achieving a convenient, fully integrated artificial pancreas system.

### SUMMARY

Drug infusion systems are disclosed including an infusion head configured to deliver a primary drug and a secondary drug to a patient's subcutaneous tissue. The secondary drug may be provided in a controlled-release material and/or a drug-containing reservoir. Methods for assembling and using such drug infusion systems are also disclosed. The secondary drug may extend the wear time of the drug infusion systems.

According to an embodiment of the present disclosure, an infusion head of a drug infusion system is disclosed, the drug infusion system including a drug delivery device configured to deliver a primary drug to the infusion head. The infusion head includes a housing including an inlet channel and an outlet channel that cooperate to form a fluid flow path for the primary drug through the housing, an inlet port coupled to the inlet channel of the housing, an infusion element coupled to the outlet channel of the housing and configured for insertion in a patient's skin, and at least one drug-loaded component inserted into the inlet channel or the outlet channel of the housing, the at least one drug-loaded component including a controlled-release material with a secondary drug on at least a surface wetted by the primary drug such that the drug-loaded component releases the secondary drug into the primary drug traveling through the fluid flow path.

According to another embodiment of the present disclosure, an infusion head of a drug infusion system is disclosed, the drug infusion system including a drug delivery device configured to deliver a primary drug to the infusion head. The infusion head includes a housing including an upper surface that faces away from a patient's skin, a lower surface that faces toward the patient's skin, and a side surface that spans between the upper surface and the lower surface, an inlet channel configured to receive the primary drug from the drug delivery device, the inlet channel having a side opening in the side surface of the housing, an outlet channel configured to receive the primary drug from the inlet channel, the outlet channel having an optional upper opening in the upper surface of the housing and a lower opening in the lower surface of the housing, an inlet port positioned in the side opening of the inlet channel, an optional septum positioned in the upper opening of the outlet channel, an infusion element positioned in the lower opening of the outlet channel and configured for insertion in the patient's skin, and at least one drug-loaded component sized for insertion through the side opening of the inlet channel or the optional upper opening of the outlet channel, the at least one drug-loaded component including a controlled-release material that releases a secondary drug into the primary drug traveling through the housing.

According to yet another embodiment of the present disclosure, an infusion head of a drug infusion system is disclosed, the drug infusion system including a drug delivery device configured to deliver a primary drug to the infusion head. The infusion head includes a first housing, a second housing coupled to the first housing, a fluid flow path for the primary drug between the first and second housings, the fluid flow path including an inlet channel, at least one microchannel in the inlet channel that lengthens the fluid flow path through the first and second housings, and an outlet channel in fluid communication with the inlet channel, and at least one drug-loaded component inserted between the first and second housings in fluid communication with the fluid flow path, the at least one drug-loaded component including a controlled-release material that releases a secondary drug into the primary drug traveling through the fluid flow path.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 is a schematic view of an exemplary drug infusion system of the present disclosure including a drug delivery device containing a primary drug, an intermediate tubing set, and an infusion head containing a secondary drug;

FIG. 2 is a partially exploded perspective view of the drug delivery device, the tubing set, and the infusion head of FIG. 1;

FIG. 3 is an exploded perspective view of the infusion head of FIG. 2 including an in-line drug-loaded bushing and a single-piece housing;

FIG. 4 is an assembled perspective view of another infusion head including an in-line drug-loaded tube and a single-piece housing;

FIG. 5 is an exploded perspective view of the infusion head of FIG. 4;

FIG. 6 is an assembled perspective view of another infusion head including in-line drug-loaded rods and a single-piece housing;

FIG. 7 is an exploded perspective view of the infusion head of FIG. 6;

FIG. 8 is an assembled perspective view of another infusion head including in-line drug-loaded pellets and a single-piece housing;

FIG. 9 is an exploded perspective view of the infusion head of FIG. 8;

FIG. 10 is an assembled perspective view of another infusion head including an in-line drug-loaded tunnel and a multi-piece housing;

FIG. 11 is an exploded perspective view of the infusion head of FIG. 10;

FIG. 12 is an assembled perspective view of another infusion head including an in-line drug-loaded plate and a multi-piece housing;

FIG. 13 is an exploded perspective view of the infusion head of FIG. 12;

FIG. 14 is an assembled perspective view of another infusion head including in-line drug-loaded rods and a multi-piece housing;

FIG. 15 is an exploded perspective view of the infusion head of FIG. 14;

FIG. 16 is an assembled perspective view of another infusion head including in-line drug-loaded pellets and a multi-piece housing;

FIG. 17 is an exploded perspective view of the infusion head of FIG. 16;

FIG. 18 is an assembled perspective view of a variant of the infusion head of FIG. 16;

FIG. 19 is an assembled perspective view of another infusion head including an in-line drug-loaded insert and a multi-piece housing;

FIG. 20 is an exploded perspective view of the infusion head of FIG. 19;

FIG. 21 is a top plan view of another infusion head including a parallel drug-containing reservoir and a single-piece housing;

FIG. 22 is a top plan view of another infusion head including a parallel drug-containing reservoir and a multi-piece housing;

FIG. 23 is a perspective view of another infusion head including an independent drug-containing reservoir and a single-piece housing;

FIG. 24 is a top plan view of another infusion head including an independent drug-containing reservoir and two independent housings;

FIG. 25 is a perspective view of another infusion head including an independent drug-containing reservoir and a double-lumen cannula;

FIG. 26 is an assembled perspective view of another infusion head including a manual drive mechanism;

FIG. 27 is an exploded perspective view of the manual drive mechanism of FIG. 26;

FIG. 28 is a cross-sectional view of the infusion head of FIG. 26 in a ready state;

FIG. 29 is a cross-sectional view of the infusion head of FIG. 26 in a driven state;

FIG. 30 is a cross-sectional view of another infusion head including a morphic drive mechanism;

FIG. 31 is a cross-sectional view of another infusion head including a chemical drive mechanism; and

FIG. 32 is a perspective view of another infusion head including an active valve drive mechanism.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

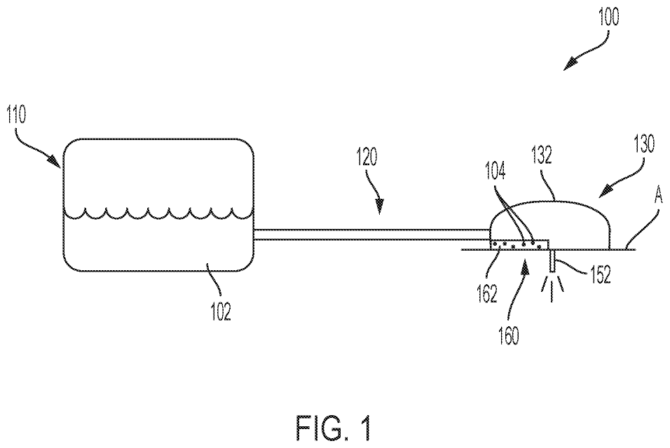

As shown schematically in FIG. 1, the present disclosure provides a drug infusion system 100 for delivering a primary drug 102 and a secondary drug 104 into a patient's subcutaneous tissue. The drug infusion system 100 includes a drug delivery device 110 containing the primary drug 102, an intermediate tubing set 120, and an infusion head 130 containing the secondary drug 104. In use, the drug infusion system 100 delivers the primary drug 102 from the drug delivery device 110, through the tubing set 120, and into the infusion head 130. The infusion head 130 includes an adhesive pad A that adheres to the patient's skin and a drug infusion element in the form of a cannula 152 or a needle that penetrates the patient's skin to deliver the primary drug 102 into the patient's subcutaneous tissue. The infusion head 130 also delivers the secondary drug 104 into the patient's subcutaneous tissue, either together with or separately from the primary drug 102.

### 1. Primary and Secondary Drugs

Referring still to FIG. 1, the primary drug 102 may be delivered from the drug delivery device 110. The primary drug 102 may include one or more therapeutic agents including, but not limited to, insulins, insulin analogs (e.g., insulin lispro, insulin glargine), insulin derivatives, GLP-1 receptor agonists (e.g., dulaglutide, liraglutide), glucagon, glucagon analogs, glucagon derivatives, gastric inhibitory polypeptides (GIP), GIP analogs, GIP derivatives, oxyntomodulin analogs, oxyntomodulin derivatives, therapeutic antibodies, and any other therapeutic agents capable of delivery by the drug delivery device 110. The primary drug 102 may be formulated with one or more excipients.

The secondary drug 104 may be delivered from the infusion head 130. The secondary drug 104 may include one or more therapeutic agents configured to promote infusion site viability. As a result, the infusion site may last longer than 3 days, 5 days, 7 days, or more, such as about 7 to 14 days, which may reduce drug waste, reduce scarring, and enable a once-weekly or once-biweekly change-over time frame for a fully integrated artificial pancreas system.

The secondary drug 104 may include one or more anti-inflammatory agents, more specifically nonsteroidal anti-inflammatory drugs (NSAIDs). Exemplary anti-inflammatory agents include meloxicam. ibuprofen, naproxen, aspirin, plumbagin, plumericin, celecoxib, diclofenac, etodolac, indomethacin, ketoprofen, ketorolac, nabumetone, oxaprozin, piroxicam, salsalate, sulindac, tolmetin, rapamycin, dexamethasone, betamethasone, heparin, sirolimus, and paxlitaxel, for example. Such anti-inflammatory agents may reduce pain, decrease fever, reduce the likelihood of blood clots, and/or decrease inflammation.

The secondary drug 104 may include glucagon, glucagon analogs, and glucagon derivatives (generally “glucagon”) to raise a patient's glucose levels (e.g., blood glucose levels or interstitial glucose levels). The glucagon may be delivered in (a) emergency dosing during a severe hypoglycemic event, (b) mini-dosing to prevent or treat impending mild hypoglycemia, and (c) algorithmic dosing for closed-loop artificial pancreas operation.

The secondary drug 104 may include other therapeutic agents, such as incretin hormones (e.g., gastric inhibitory polypeptide (GIP) and glucagon-like peptide-1 (GLP-1)), inhibitors of tyrosine kinase (e.g., masitinib), inhibitors of the matricellular protein Thrombospondin 2 (TSP2), inhibitors of fibrosis-stimulating cytokines including Connective Tissue Growth Factor (CTGF), inhibitors of members of the integrin family of receptors, Vascular Endothelial Growth Factor (VEGF), antimicrobial agents (e.g., silver) and diffusion enhancing agents (e.g., hyaluronidase), for example. In one particular example, the secondary drug 104 includes the therapeutic agent VEGF in combination with the anti-inflammatory agent dexamethasone, but other combinations are also contemplated.

In certain embodiments, the secondary drug 104 may be part of a controlled-release material 160. As shown in FIG. 1, for example, the controlled-release material 160 may include a matrix 162 impregnated with the secondary drug 104. The matrix 162 may degrade or otherwise release the secondary drug 104 in a controlled manner when wetted by an appropriate fluid, such as the primary drug 102, the patient's bodily fluids, or another suitable fluid. Exemplary degradable polymers for use as the matrix 162 include polyethylene glycol (PEG), polyvinyl alcohol (PVA), ethylene vinyl acetate (EVA) polylactic acid (PLA), polyglycolic acid (PGA), poly(lactic-co-glycolic acid) (PLGA), polyhydroxyethylmethacrylate (PHEMA), poly(methacrylic acid) (PMAA), alginate, (poly) phosphoryl chlorines and (poly) ester amides, for example. The release of the secondary drug 104 from the controlled-release material 160 may be controlled by modifying the surface area of the controlled-release material 160 that is exposed to the wetting fluid, the flow rate of the wetting fluid, and other variables. In addition to controlling release of the secondary drug 104, the controlled-release material 160 may also improve film or coating properties of the secondary drug 104, improve solubility of the secondary drug 104, and/or improve elution properties of the secondary drug 104.

As described further below, the secondary drug 104 may be used in various ways throughout the drug infusion system 100, particularly the infusion head 130. The type, structure, shape, size, location, and other properties of the secondary drug 104 may vary. When the secondary drug 104 is part of the controlled-release material 160, for example, various components of the infusion head 130, in whole or in part, may be manufactured from (e.g., molded from) the controlled-release material 160, coated with the controlled-release material 160, filled with the controlled-release material 160, or otherwise processed to contain the controlled-release material 160. Such components that release the secondary drug 104 from the controlled-release material 160 may be referred to herein as “drug-loaded” components.

### 2. Drug Infusion System

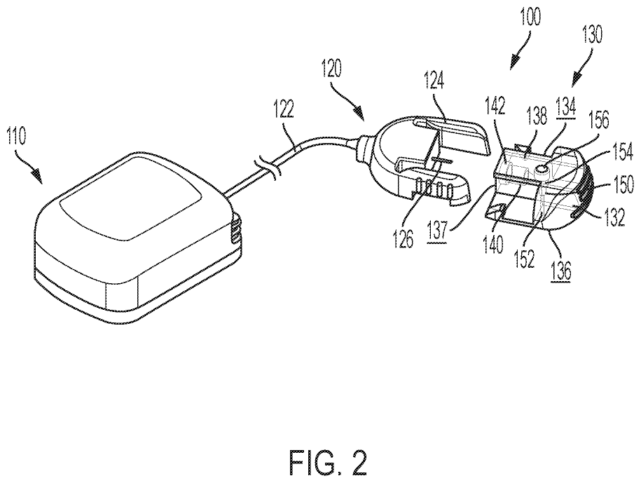

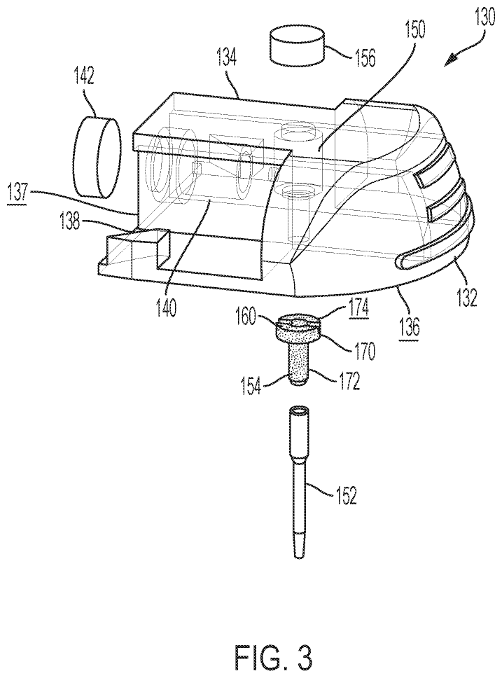

The drug infusion system 100 will now be described further with respect to FIGS. 2 and 3.

The drug delivery device 110 of the drug infusion system 100 is configured to deliver the primary drug 102 (FIG. 1). The illustrative drug delivery device 110 is a pump, but it is also within the scope of the present disclosure for the drug delivery device 110 to be a bolus injector, autoinjector, injection pen, or another suitable drug delivery device. The drug delivery device 110 may be controlled via built-in controls (e.g., a touchscreen, buttons) or via wireless controls (e.g., the patient's smartphone). In certain embodiments, a set of multiple drug delivery devices 110 may be provided within the drug infusion system 100, with each drug delivery device 110 containing a different volume of the primary drug 102, such as 1 mL to 20 mL or more, for example.

The tubing set 120 of the drug infusion system 100 may be removably coupled between the drug delivery device 110 and the infusion head 130. The tubing set 120 includes a flexible line set tubing 122. At the end adjacent to the infusion head 130, the illustrative tubing set 120 also includes a male-shaped connector 124 and a needle port 126.

The infusion head 130 of the drug infusion system 100 is configured to deliver the secondary drug 104 (FIG. 1). The illustrative infusion head 130 includes a single-piece or multi-piece housing 132. The housing 132 may be formed from plastic or another suitable material. The housing 132 includes an upper surface 134 that faces away from the patient's skin, a lower surface 136 that faces toward the patient's skin, and a side surface 137 that spans between the upper surface 134 and the lower surface 136. The lower surface 136 may support the adhesive pad A (FIG. 1) for adhering the infusion head 130 to the patient's skin. The infusion head 130 also includes a female-shaped connector 138 that couples with the male-shaped connector 124 of the tubing set 120.

The infusion head 130 further includes a horizontal, inlet channel 140 and a vertical, outlet channel 150 in the housing 132. The channels 140, 150 may be molded, drilled, or otherwise formed in the housing 132. As shown in FIGS. 2 and 3, the inlet channel 140 is open to the side surface 137 of the housing 132, and the outlet channel 150 is open to both the upper surface 134 and the lower surface 136 of the housing 132. Within the side opening to the inlet channel 140 in the side surface 137 of the housing 132, the infusion head 130 includes an inlet port in the form of a septum port 142 configured to receive the needle port 126 of the tubing set 120 in a sealed manner. Within the lower opening to the outlet channel 150 in the lower surface 136 of the housing 132, the infusion head 130 includes the cannula 152 that extends past the housing 132 and into the patient's skin and a bushing that supports the cannula 152. Within the upper opening to the outlet channel 150 in the upper surface 134 of the housing 132, the infusion head 130 includes an introducer septum 156 that closes the outlet channel 150 and is configured to receive an introducer needle (not shown) that pierces the patient's skin for receipt of the cannula 152.

Although the illustrated infusion head 130 includes a cannula 152 as the infusion element, it is also within the scope of the present disclosure for the infusion head 130 to include a needle as the introducer element. In this embodiment, a separate introducer needle would not be required. Thus, the infusion head 130 need not include the upper opening of the outlet channel 150 or the introducer septum 156 located therein.

### 3. In-Line Drug-Loaded Bushing with Single-Piece Housing

Referring next to FIG. 3, the bushing 154 of the infusion head 130 contains the controlled-release material 160 with the secondary drug 104 (FIG. 1). The drug-loaded bushing 154 is positioned in the outlet channel 150 and is therefore in line (i.e., in series) with the flow path of the primary drug 102 (FIG. 1) through the inlet channel 140 and the outlet channel 150 of the infusion head 130.

The drug-loaded bushing 154 of FIG. 3 includes a head 170 disposed above the cannula 152 and a hollow stem 172 that extends into the cannula 152. The head 170 and/or the stem 172 of the drug-loaded bushing 154 may contain the controlled-release material 160. More specifically, and as indicated above, the head 170 and/or the stem 172 of the drug-loaded bushing 154 may be manufactured from (e.g., molded from) the controlled-release material 160, coated with the controlled-release material 160, filled with the controlled-release material 160, or otherwise processed to contain the controlled-release material 160. In one embodiment, at least the upper surface 174 of the head 170 and the interior of the hollow stem 172 that are wetted by the primary drug 102 (FIG. 1) may contain the controlled-release material 160.

The infusion head 130 may be assembled by inserting the drug-loaded bushing 154 into the housing 132. More specifically, the infusion head 130 may be assembled by press fitting the cannula 152 onto the drug-loaded bushing 154, dropping the cannula 152 and the drug-loaded bushing 154 into the upper end of the outlet channel 150, inserting the introducer septum 156 into the upper surface 134 of the housing 132 to seal the upper end of the outlet channel 150, and heat-staking or otherwise coupling the introducer septum 156 into place. Advantageously, except for the insertion of the drug-loaded bushing 154 through the outlet channel 150, this assembly method may be similar to existing assembly methods.

In use, the primary drug 102 (FIG. 1) that travels across and through the drug-loaded bushing 154 in the outlet channel 150 may cause the secondary drug 104 (FIG. 1) to be released from the controlled-release material 160. The primary drug 102 and the secondary drug 104 may exit the infusion head 130 together through the same cannula 152, which minimizes the number of needle sticks to the patient.

### 4. In-Line Drug-Loaded Tube with Single-Piece Housing

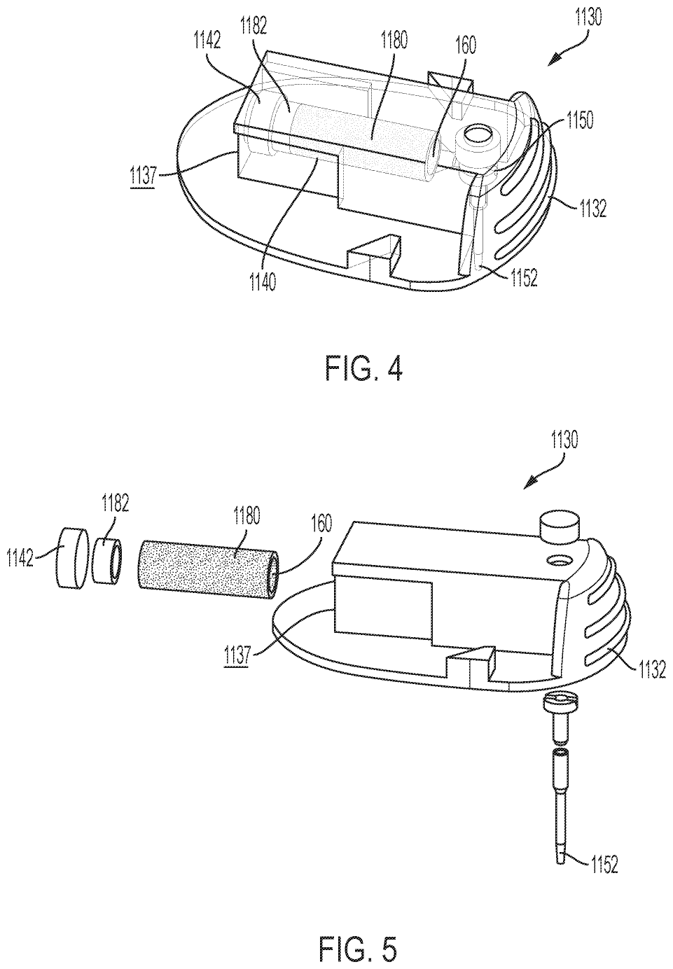

Referring next to FIGS. 4 and 5, another infusion head 1130 is provided for use with the drug delivery system 100 of FIG. 2. The infusion head 1130 may be similar to the above-described infusion head 130, with like reference numerals identifying like elements, except as described below.

The infusion head 1130 includes a single-piece housing 1132, an inlet channel 1140, and an outlet channel 1150. Within the inlet channel 1140, the infusion head 1130 includes a drug-loaded tube 1180 containing the controlled-release material 160 with the secondary drug 104 (FIG. 1) and an optional funnel 1182. The drug-loaded tube 1180 in the inlet channel 1140 is positioned in line with the flow path of the primary drug 102 (FIG. 1) through the inlet channel 1140 and the outlet channel 1150 of the infusion head 1130.

As indicated above, the drug-loaded tube 1180 may be manufactured from (e.g., molded from) the controlled-release material 160, coated with the controlled-release material 160, filled with the controlled-release material 160, or otherwise processed to contain the controlled-release material 160. In one embodiment, at least the interior surface of the drug-loaded tube 1180 that is wetted by the primary drug 102 (FIG. 1) may contain the controlled-release material 160.

The surface area of the drug-loaded tube 1180 may vary to achieve desired contact with the primary drug 102 (FIG. 1). The length of the drug-loaded tube 1180 may vary. The illustrative drug-loaded tube 1180 is a relatively long cylinder, but the drug-loaded tube 1180 may also be a relatively short ring or disc, for example. The thickness of the drug-loaded tube 1180 may also vary. The illustrative drug-loaded tube 1180 has a relatively thick wall, which may be formed by extrusion or by hollowing (e.g., drilling, punching) a solid cylinder. However, the drug-loaded tube 1180 may also have a relatively thin wall, which may be formed by rolling a sheet of material.

The infusion head 1130 may be assembled by inserting the drug-loaded tube 1180 into the housing 1132. More specifically, the infusion head 1130 may be assembled by inserting the drug-loaded tube 1180 sideways into the side opening of the inlet channel 1140, optionally inserting the funnel 1182 into the side opening of the inlet channel 1140, inserting the septum port 1142 into the side surface 1137 of the housing 1132 to seal the side opening of the inlet channel 1140, and heat-staking or otherwise coupling the septum port 1142 into place. The optional funnel 1182 may ensure a fluid-tight connection between the septum port 1142 and the drug-loaded tube 1180. Other elements of the infusion head 1130, including the cannula 1152, may be assembled in a similar manner as described above with respect to infusion head 130. Advantageously, except for the insertion of the drug-loaded tube 1180 and the funnel 1182 through the side opening of the inlet channel 1140, this assembly method may be similar to existing assembly methods.

In use, the primary drug 102 (FIG. 1) that travels through the drug-loaded tube 1180 in the inlet channel 1140 may cause the secondary drug 104 (FIG. 1) to be released from the controlled-release material 160. The primary drug 102 and the secondary drug 104 may exit the infusion head 1130 together through the same cannula 1152, which minimizes the number of needle sticks to the patient.

### 5. In-Line Drug-Loaded Rods with Single-Piece Housing

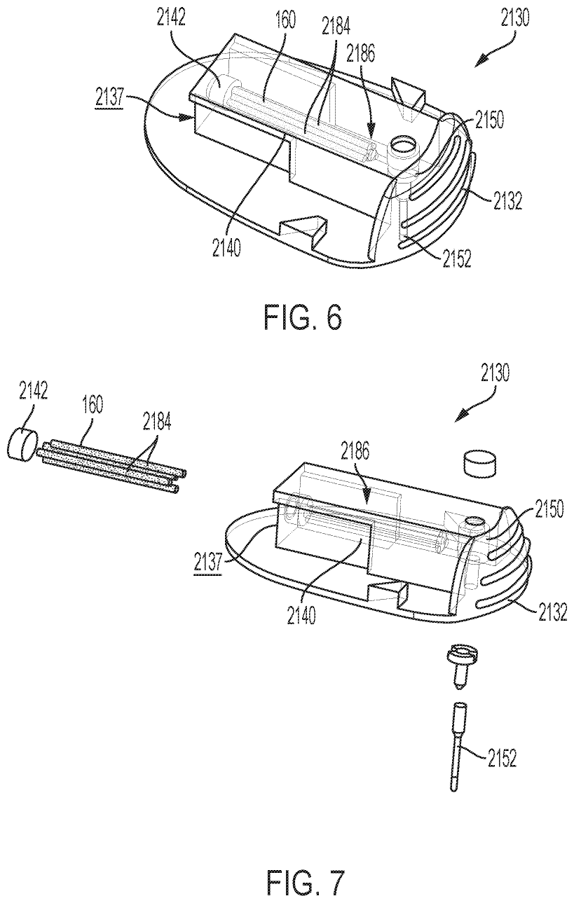

Referring next to FIGS. 6 and 7, another infusion head 2130 is provided for use with the drug delivery system 100 of FIG. 2. The infusion head 2130 may be similar to the above-described infusion heads 130, 1130, with like reference numerals identifying like elements, except as described below.

The infusion head 2130 includes a single-piece housing 2132, an inlet channel 2140, and an outlet channel 2150. Within the inlet channel 2140, the infusion head 2130 includes one or more drug-loaded rods 2184 containing the controlled-release material 160 with the secondary drug 104 (FIG. 1). The inlet channel 2140 may be sized and shaped to match the drug-loaded rods 2184. As shown in FIG. 7, for example, the inlet channel 2140 includes grooves 2186, with each groove 2186 being sized and shaped to retain a corresponding drug-loaded rod 2184 in a desired location. The drug-loaded rods 2184 in the inlet channel 2140 are positioned side-by-side and in line with the flow path of the primary drug 102 (FIG. 1) through the inlet channel 2140 and the outlet channel 2150 of the infusion head 2130. It is also within the scope of the present disclosure for the drug-loaded rods 2184 to be positioned end-to-end or in other arrangements.

As indicated above, each drug-loaded rod 2184 may be manufactured from (e.g., molded from) the controlled-release material 160, coated with the controlled-release material 160, filled with the controlled-release material 160, or otherwise processed to contain the controlled-release material 160. In one embodiment, at least the exterior surface of each drug-loaded rod 2184 that is wetted by the primary drug 102 (FIG. 1) may contain the controlled-release material 160.

The surface area of the drug-loaded rods 2184 may vary to achieve desired contact with the primary drug 102 (FIG. 1). The length of each drug-loaded rod 2184 may vary. The illustrative drug-loaded rods 2184 are relatively long cylinders, but the drug-loaded rods 2184 may also be a relatively short pegs, for example. The diameter of each drug-loaded rod 2184 may also vary. The illustrative infusion head 2130 includes four drug-loaded rods 2184, but the infusion head 2130 may also include a smaller number of thicker rods 2184 or a larger number of thinner rods 2184, for example.

The infusion head 2130 may be assembled by inserting the drug-loaded rods 2184 into the housing 2132. More specifically, the infusion head 1130 may be assembled by inserting the drug-loaded rods 2184 sideways into the corresponding grooves 2186 of the inlet channel 2140 through the side opening of the inlet channel 2140, inserting the septum port 2142 into the side surface 2137 of the housing 2132 to seal the side opening of the inlet channel 2140, and heat-staking or otherwise coupling the septum port 2142 into place. Other elements of the infusion head 2130, including the cannula 2152, may be assembled in a similar manner as described above with respect to infusion head 130. Advantageously, except for the insertion of the drug-loaded rods 2184 through the side opening of the inlet channel 2140, this assembly method may be similar to existing assembly methods.

In use, the primary drug 102 (FIG. 1) that travels across the drug-loaded rods 2184 in the inlet channel 2140 may cause the secondary drug 104 (FIG. 1) to be released from the controlled-release material 160. The primary drug 102 and the secondary drug 104 may exit the infusion head 2130 together through the same cannula 2152, which minimizes the number of needle sticks to the patient.

### 6. In-Line Drug-Loaded Pellets with Single-Piece Housing

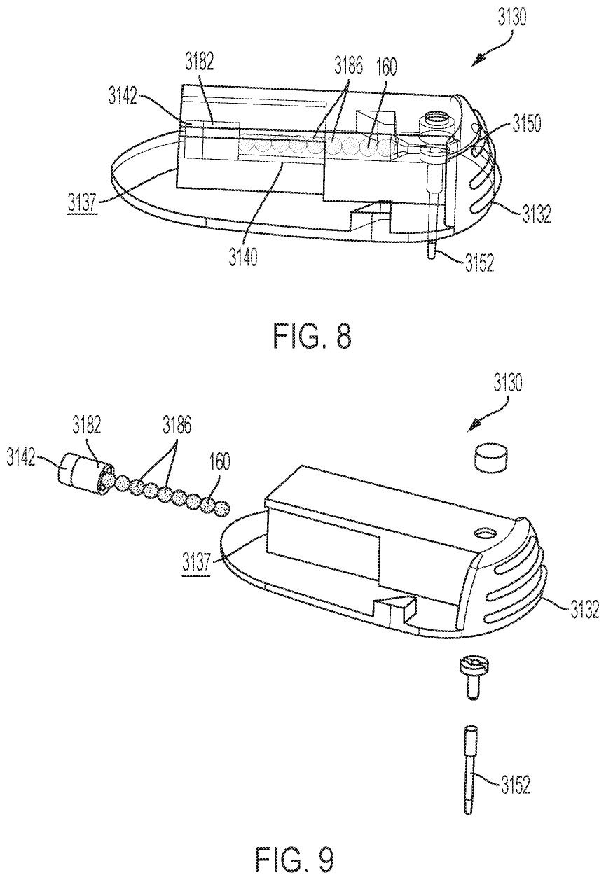

Referring next to FIGS. 8 and 9, another infusion head 3130 is provided for use with the drug delivery system 100 of FIG. 2. The infusion head 3130 may be similar to the above-described infusion heads 130, 1130, 2130, with like reference numerals identifying like elements, except as described below.

The infusion head 3130 includes a single-piece housing 3132, an inlet channel 3140, and an outlet channel 3150. Within the inlet channel 3140, the infusion head 3130 includes one or more drug-loaded pellets 3186 containing the controlled-release material 160 with the secondary drug 104 (FIG. 1) and an optional funnel 3182. The drug-loaded pellets 3186 in the inlet channel 3140 are positioned in line with the flow path of the primary drug 102 (FIG. 1) through the inlet channel 3140 and the outlet channel 3150 of the infusion head 3130.

As indicated above, each drug-loaded pellet 3186 may be manufactured from (e.g., molded from) the controlled-release material 160, coated with the controlled-release material 160, filled with the controlled-release material 160, or otherwise processed to contain the controlled-release material 160. In one embodiment, at least the exterior surface of each drug-loaded pellet 3186 that is wetted by the primary drug 102 (FIG. 1) may contain the controlled-release material 160.

The surface area of the drug-loaded pellets 3186 may vary to achieve desired contact with the primary drug 102 (FIG. 1). The size and number of the drug-loaded pellets 3186 may vary. The illustrative infusion head 3130 includes nine uniformly-sized, tightly-packed pellets 3186, but the pellets 3186 may be non-uniform in size and/or more loosely distributed within the inlet channel 3140.

The infusion head 3130 may be assembled by inserting the drug-loaded pellets 3186 into the housing 3132. More specifically, the infusion head 3130 may be assembled by inserting the drug-loaded pellets 3186 sideways into the side opening of the inlet channel 3140, optionally inserting the funnel 3182 into the side opening of the inlet channel 3140, inserting the septum port 3142 into the side surface 3137 of the housing 3132 to seal the side opening of the inlet channel 3140, and heat-staking or otherwise coupling the septum port 3142 into place. The optional funnel 3182 may ensure a fluid-tight connection between the septum port 3142 and the drug-loaded pellets 3186. Other elements of the infusion head 3130, including the cannula 3152, may be assembled in a similar manner as described above with respect to infusion head 130. Advantageously, except for the insertion of the drug-loaded pellets 3186 and funnel 3182 through side opening of the inlet channel 3140, this assembly method may be similar to existing assembly methods.

In use, the primary drug 102 (FIG. 1) that travels across the drug-loaded pellets 3186 in the inlet channel 3140 may cause the secondary drug 104 (FIG. 1) to be released from the controlled-release material 160. The primary drug 102 and the secondary drug 104 may exit the infusion head 3130 together through the same cannula 3152, which minimizes the number of needle sticks to the patient.

### 7. In-Line Drug-Loaded Tunnel with Multi-Piece Housing

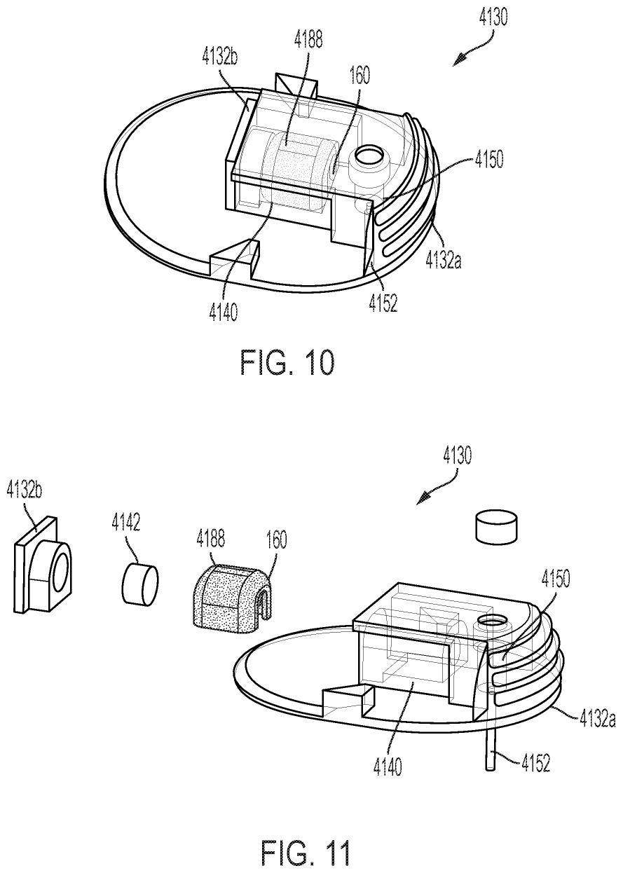

Referring next to FIGS. 10 and 11, another infusion head 4130 is provided for use with the drug delivery system 100 of FIG. 2. The infusion head 4130 may be similar to the above-described infusion heads 130, 1130, 2130, 3130, with like reference numerals identifying like elements, except as described below.

The infusion head 4130 includes a multi-piece housing, more specifically a main housing 4132a and a side housing 4132b, an inlet channel 4140, and an outlet channel 4150. Within the inlet channel 4140, the infusion head 4130 includes a drug-loaded tunnel 4188 containing the controlled-release material 160 with the secondary drug 104 (FIG. 1). The inlet channel 4140 may be cooperatively defined by the housings 4132a, 4132b and may be sized and shaped to match the U-shaped drug-loaded tunnel 4188, as shown in FIG. 11. The drug-loaded tunnel 4188 in the inlet channel 4140 is positioned in line with the flow path of the primary drug 102 (FIG. 1) through the inlet channel 4140 and the outlet channel 4150 of the infusion head 4130.

As indicated above, the drug-loaded tunnel 4188 may be manufactured from (e.g., molded from) the controlled-release material 160, coated with the controlled-release material 160, filled with the controlled-release material 160, or otherwise processed to contain the controlled-release material 160. In one embodiment, at least the interior surface of the drug-loaded tunnel 4188 that is wetted by the primary drug 102 (FIG. 1) may contain the controlled-release material 160.

The infusion head 4130 may be assembled by inserting the drug-loaded tunnel 4188 into the housing 4132a, 4132b. More specifically, the infusion head 4130 may be assembled by inserting the drug-loaded tunnel 4188 sideways into the side opening of the inlet channel 4140 in the main housing 4132a, inserting the septum port 4142 into the side housing 4132b to seal the side opening of the inlet channel 4140, and then coupling (e.g., laser welding, ultrasonic welding, thermal bonding) the side housing 4132b to the main housing 4132a in a sealed manner to capture the drug-loaded tunnel 4188. Other elements of the infusion head 4130, including the cannula 4152, may be assembled in a similar manner as described above with respect to infusion head 130.

In use, the primary drug 102 (FIG. 1) that travels through the drug-loaded tunnel 4188 in the inlet channel 4140 may cause the secondary drug 104 (FIG. 1) to be released from the controlled-release material 160. The primary drug 102 and the secondary drug 104 may exit the infusion head 4130 together through the same cannula 4152, which minimizes the number of needle sticks to the patient.

### 8. In-Line Drug-Loaded Plate with Multi-Piece Housing

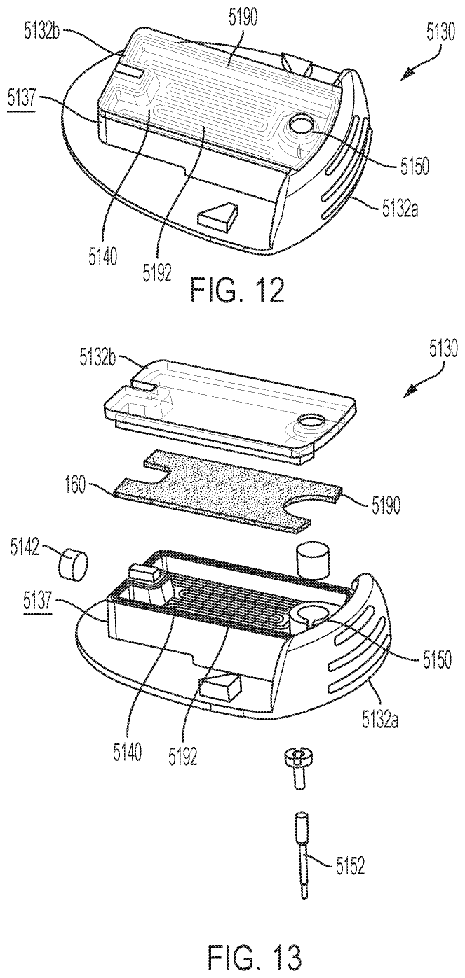

Referring next to FIGS. 12 and 13, another infusion head 5130 is provided for use with the drug delivery system 100 of FIG. 2. The infusion head 5130 may be similar to the above-described infusion heads 130, 1130, 2130, 3130, 4130, with like reference numerals identifying like elements, except as described below.

The infusion head 5130 includes a multi-piece housing, more specifically a main housing 5132a and an upper housing 5132b, an inlet channel 5140, and an outlet channel 5150. Within the inlet channel 5140, the infusion head 5130 includes a drug-loaded plate 5190 containing the controlled-release material 160 with the secondary drug 104 (FIG. 1). The inlet channel 5140 may be cooperatively defined by the housings 5132a, 5132b and may be sized and shaped to match the H-shaped drug-loaded plate 5190, as shown in FIG. 13. The drug-loaded plate 5190 in the inlet channel 5140 is positioned in line with the flow path of the primary drug 102 (FIG. 1) through the inlet channel 5140 and the outlet channel 5150 of the infusion head 5130.

As indicated above, the drug-loaded plate 5190 may be manufactured from (e.g., molded from) the controlled-release material 160, coated with the controlled-release material 160, filled with the controlled-release material 160, or otherwise processed to contain the controlled-release material 160. In one embodiment, at least the lower surface of the drug-loaded plate 5190 that is wetted by the primary drug 102 (FIG. 1) may contain the controlled-release material 160.

In certain embodiments, the inlet channel 5140 includes one or more microchannels 5192 to increase the effective length of the inlet channel 5140 and the time of exposure to the adjacent drug-loaded plate 5190. In FIG. 13, for example, the main housing 5132a includes a serpentine-shaped microchannel 5192 beneath the drug-loaded plate 5190 to encourage a serpentine flow pattern through the inlet channel 5140. The microchannel 5192 may have different arrangements to encourage different flow patterns, such as spiral arrangements, zig-zag arrangements, or other arrangements. If necessary, a seal (not shown) may be provided around the drug-loaded plate 5190 to retain the fluid in the microchannel 5192 and prevent leakage above the drug-loaded plate 5190. However, in other embodiments, the microchannel 5192 may be sized and shaped to provide the path of least resistance, thereby discouraging leakage above the drug-loaded plate 5190.

The infusion head 5130 may be assembled by inserting the drug-loaded plate 5190 into the housings 5132a, 5132b. More specifically, the infusion head 5130 may be assembled by inserting the drug-loaded plate 5190 downward onto the microchannel 5192 of the main housing 5132a, coupling (e.g., laser welding, ultrasonic welding, thermal bonding) the upper housing 5132b to the main housing 5132a in a sealed manner to capture the drug-loaded plate 5190 and define the inlet channel 5140, inserting the septum port 5142 into the side surface 5137 of the main housing 5132a to seal the side opening of the inlet channel 5140, and heat-staking or otherwise coupling the septum port 5142 to the main housing 5132a. Other elements of the infusion head 5130, including the cannula 5152, may be assembled in a similar manner as described above with respect to infusion head 130.

In use, the primary drug 102 (FIG. 1) that travels across the drug-loaded plate 5190 in the inlet channel 5140 may cause the secondary drug 104 (FIG. 1) to be released from the controlled-release material 160. The microchannel 5192 in the inlet channel 5140 may increase the time of exposure between the primary drug 102 and the drug-loaded plate 5190, thereby increasing the concentration of the secondary drug 104 in the primary drug 102. The primary drug 102 and the secondary drug 104 may exit the infusion head 5130 together through the same cannula 5152, which minimizes the number of needle sticks to the patient.

### 9. In-Line Drug-Loaded Rods with Multi-Piece Housing

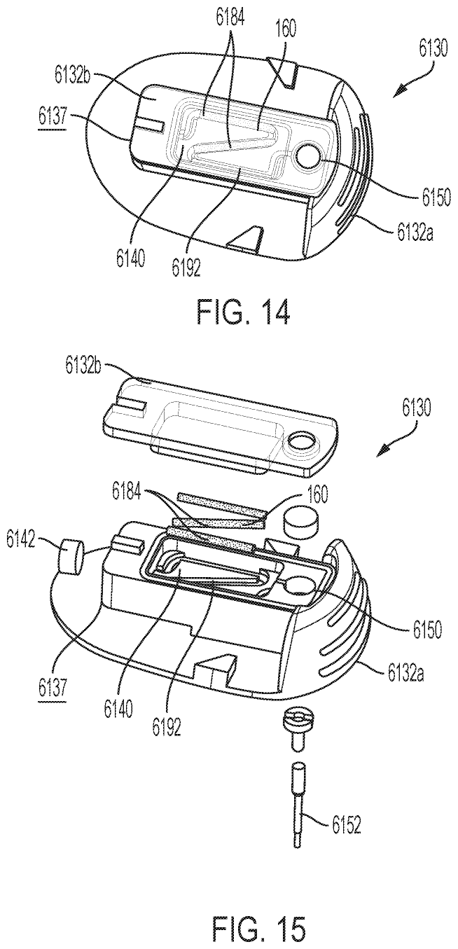

Referring next to FIGS. 14 and 15, another infusion head 6130 is provided for use with the drug delivery system 100 of FIG. 2. The infusion head 6130 may be similar to the above-described infusion heads 130, 1130, 2130, 3130, 4130, 5130, with like reference numerals identifying like elements, except as described below.

The infusion head 6130 includes a multi-piece housing, more specifically a main housing 6132a and an upper housing 6132b, an inlet channel 6140, and an outlet channel 6150. Within the inlet channel 6140, the infusion head 6130 includes drug-loaded rods 6184 containing the controlled-release material 160 with the secondary drug 104 (FIG. 1). The inlet channel 6140 may be cooperatively defined by the housings 6132a, 6132b, as shown in FIG. 15. The drug-loaded rods 6184 in the inlet channel 6140 are positioned end-to-end and in line with the flow path of the primary drug 102 (FIG. 1) through the inlet channel 6140 and the outlet channel 6150 of the infusion head 6130. It is also within the scope of the present disclosure for the drug-loaded rods 6184 to be positioned side-by-side or in other arrangements.

As indicated above, the drug-loaded rods 6184 may be manufactured from (e.g., molded from) the controlled-release material 160, coated with the controlled-release material 160, filled with the controlled-release material 160, or otherwise processed to contain the controlled-release material 160. In one embodiment, at least the exterior surface of each drug-loaded rod 6184 that is wetted by the primary drug 102 (FIG. 1) may contain the controlled-release material 160.

In certain embodiments, the inlet channel 6140 includes one or more microchannels 6192 to increase the effective length of the inlet channel 6140 and the time of exposure to the drug-loaded rods 6184. The microchannel 6192 may also be sized to retain the drug-loaded rods 6184 in their desired locations. In FIG. 15, for example, the main housing 6132a includes a Z-shaped microchannel 6192 to encourage a Z-shaped flow pattern through the inlet channel 6140 across the drug-loaded rods 6184. The microchannel 6192 may have different arrangements to encourage different flow patterns, such as spiral arrangements, zig-zag arrangements, or other arrangements.

The infusion head 6130 may be assembled by inserting the drug-loaded rods 6184 into the housings 6132a, 6132b. More specifically, the infusion head 6130 may be assembled by inserting the drug-loaded rods 6184 downward into the microchannel 6192 of the main housing 6132a, coupling (e.g., laser welding, ultrasonic welding, thermal bonding) the upper housing 6132b to the main housing 6132a in a sealed manner to capture the drug-loaded rods 6184 and define the inlet channel 6140, inserting the septum port 6142 into the side surface 6137 of the main housing 6132a to seal the side opening of the inlet channel 6140, and heat-staking or otherwise coupling the septum port 6142 to the main housing 6132a. Other elements of the infusion head 6130, including the cannula 6152, may be assembled in a similar manner as described above with respect to infusion head 130.

In use, the primary drug 102 (FIG. 1) that travels across the drug-loaded rods 6184 in the inlet channel 6140 may cause the secondary drug 104 (FIG. 1) to be released from the controlled-release material 160. The microchannel 6192 in the inlet channel 6140 may increase the time of exposure between the primary drug 102 and the drug-loaded rods 6184, thereby increasing the concentration of the secondary drug 104 in the primary drug 102. The primary drug 102 and the secondary drug 104 may exit the infusion head 6130 together through the same cannula 6152, which minimizes the number of needle sticks to the patient.

### 10. In-Line Drug-Loaded Pellets with Multi-Piece Housing

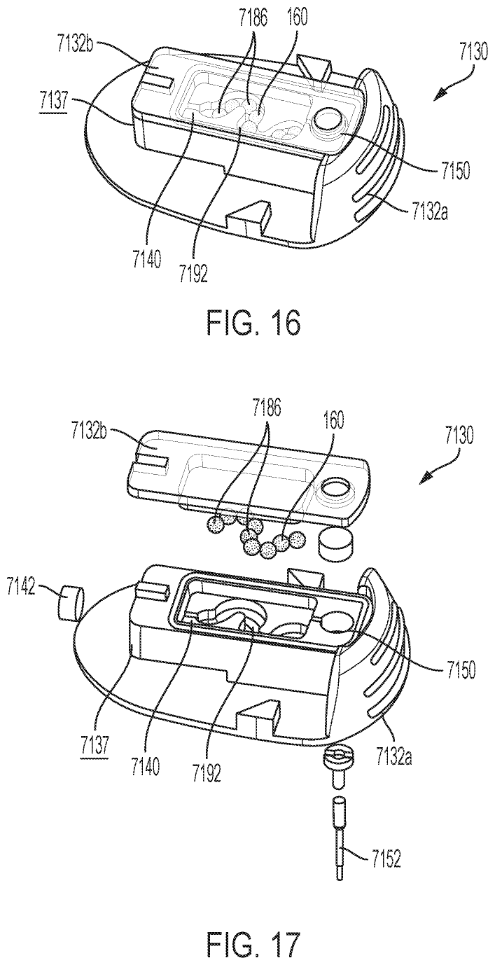

Referring next to FIGS. 16 and 17, another infusion head 7130 is provided for use with the drug delivery system 100 of FIG. 2. The infusion head 7130 may be similar to the above-described infusion heads 130, 1130, 2130, 3130, 4130, 5130, 6130, with like reference numerals identifying like elements, except as described below.

The infusion head 7130 includes a multi-piece housing, more specifically a main housing 7132a and an upper housing 7132b, an inlet channel 7140, and an outlet channel 7150. Within the inlet channel 7140, the infusion head 7130 includes drug-loaded pellets 7186 containing the controlled-release material 160 with the secondary drug 104 (FIG. 1). The inlet channel 7140 may be cooperatively defined by the housings 7132a, 7132b, as shown in FIG. 17. The drug-loaded pellets 7186 in the inlet channel 7140 are positioned in line with the flow path of the primary drug 102 (FIG. 1) through the inlet channel 7140 and the outlet channel 7150 of the infusion head 7130.

As indicated above, the drug-loaded pellets 7186 may be manufactured from (e.g., molded from) the controlled-release material 160, coated with the controlled-release material 160, filled with the controlled-release material 160, or otherwise processed to contain the controlled-release material 160. In one embodiment, at least the exterior surface of each drug-loaded pellet 7186 that is wetted by the primary drug 102 (FIG. 1) may contain the controlled-release material 160.

In certain embodiments, the inlet channel 7140 includes one or more microchannels 7192 to increase the effective length of the inlet channel 7140 and the time of exposure to the drug-loaded pellets 7186. The microchannel 7192 may also be sized to retain the drug-loaded pellets 7186 in their desired locations. In FIG. 17, for example, the main housing 7132a includes a serpentine-shaped microchannel 7192 to encourage a serpentine-shaped flow pattern through the inlet channel 6140 across the drug-loaded pellets 7186. The microchannel 7192 may have different arrangements to encourage different flow patterns, such as spiral arrangements, zig-zag arrangements, or other arrangements.

The infusion head 7130 may be assembled by inserting the drug-loaded pellets 7186 into the housings 7132a, 7132b. More specifically, the infusion head 7130 may be assembled by inserting the drug-loaded pellets 7186 downward into the microchannel 7192 of the main housing 7132a, coupling (e.g., laser welding, ultrasonic welding, thermal bonding) the upper housing 7132b to the main housing 7132a in a sealed manner to capture the drug-loaded pellets 7186 and define the inlet channel 7140, inserting the septum port 7142 into the side surface 7137 of the main housing 7132a to seal the side opening of the inlet channel 7140, and heat-staking or otherwise coupling the septum port 7142 to the main housing 7132a. Other elements of the infusion head 7130, including the cannula 7152, may be assembled in a similar manner as described above with respect to infusion head 130.

In use, the primary drug 102 (FIG. 1) that travels across the drug-loaded pellets 7186 in the inlet channel 7140 may cause the secondary drug 104 (FIG. 1) to be released from the controlled-release material 160. The microchannel 7192 in the inlet channel 7140 may increase the time of exposure between the primary drug 102 and the drug-loaded pellets 7186, thereby increasing the concentration of the secondary drug 104 in the primary drug 102. The primary drug 102 and the secondary drug 104 may exit the infusion head 7130 together through the same cannula 7152, which minimizes the number of needle sticks to the patient.

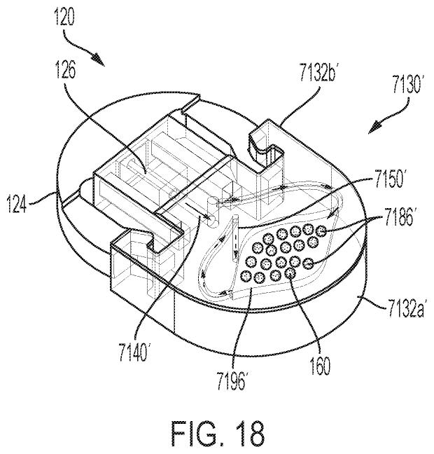

Referring next to FIG. 18, a variation of infusion head 7130′ is shown. The infusion head 7130′ includes a multi-piece housing, more specifically a main housing 7132a′ and an upper housing 7132b′, an inlet channel 7140′, and an outlet channel 7150′. The inlet channel 7140′ widens to form a reservoir 7196′ containing the drug-loaded pellets 7186′. The drug-loaded pellets 7186′ may be free to move in the reservoir 7196′ and may behave as a fluidized bed when the primary drug 102 (FIG. 1) flows through the inlet channel 7140′ and the outlet channel 7150′.

### 11. In-Line Drug-Loaded Insert with Multi-Piece Housing

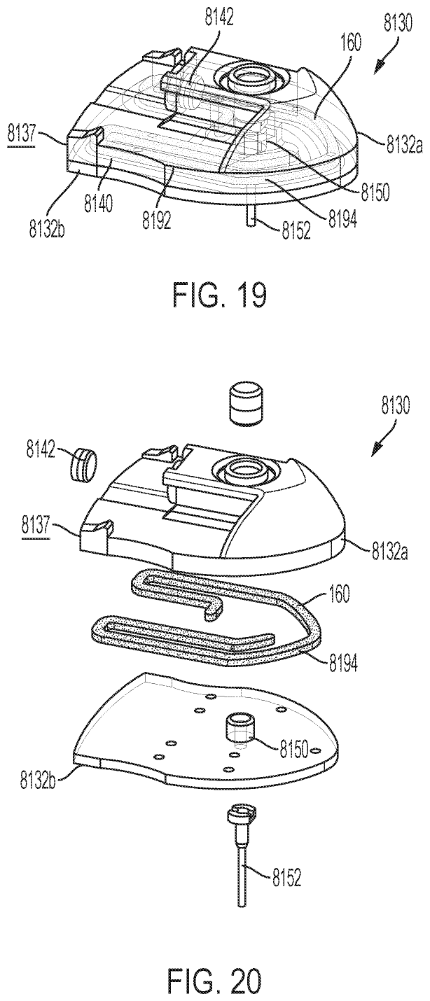

Referring next to FIGS. 19 and 20, another infusion head 8130 is provided for use with the drug delivery system 100 of FIG. 2. The infusion head 8130 may be similar to the above-described infusion heads 130, 1130, 2130, 3130, 4130, 5130, 6130, 7130 and their variants, with like reference numerals identifying like elements, except as described below.

The infusion head 8130 includes a multi-piece housing, more specifically a main housing 8132a and a lower housing 8132b, an inlet channel 8140, and an outlet channel 8150. Within the inlet channel 8140, the infusion head 8130 includes a non-linear, serpentine-shaped, drug-loaded insert 8194 having a U-shaped profile and containing the controlled-release material 160 with the secondary drug 104 (FIG. 1). The inlet channel 8140 may be cooperatively defined by the housings 8132a, 8132b, as shown in FIG. 18. The drug-loaded insert 8194 in the inlet channel 8140 is positioned in line with the flow path of the primary drug 102 (FIG. 1) through the inlet channel 8140 and the outlet channel 8150 of the infusion head 8130.

As indicated above, the drug-loaded insert 8194 may be manufactured from (e.g., molded from) the controlled-release material 160, coated with the controlled-release material 160, filled with the controlled-release material 160, or otherwise processed to contain the controlled-release material 160. In one embodiment, at least the exterior surface of the drug-loaded insert 8194 that is wetted by the primary drug 102 (FIG. 1) may contain the controlled-release material 160.

In certain embodiments, the inlet channel 8140 includes one or more microchannels 8192 to increase the effective length of the inlet channel 8140 and the time of exposure to the drug-loaded insert 8194. The microchannel 8192 may also be sized and shaped to match the drug-loaded insert 8194. In FIG. 19, for example, the main housing 8132a includes a serpentine-shaped microchannel 8192 with a U-shaped profile to encourage a serpentine-shaped flow pattern through the inlet channel 8140 across the drug-loaded insert 8194. The microchannel 8192 may have different arrangements to encourage different flow patterns, such as spiral arrangements, zig-zag arrangements, or other arrangements.

The infusion head 8130 may be assembled by inserting the drug-loaded insert 8194 into the housings 8132a, 8132b. More specifically, the infusion head 8130 may be assembled by inserting the drug-loaded insert 8194 upward into the microchannel 8192 of the main housing 8132a, coupling (e.g., laser welding, ultrasonic welding, thermal bonding) the lower housing 8132b to the main housing 8132a in a sealed manner to capture the drug-loaded insert 8194 and define the inlet channel 8140, inserting the septum port 8142 into the side surface 8137 of the main housing 8132a to seal the side opening of the inlet channel 8140, and heat-staking or otherwise coupling the septum port 8142 to the main housing 8132a. Other elements of the infusion head 8130, including the cannula 8152, may be assembled in a similar manner as described above with respect to infusion head 130.

In use, the primary drug 102 (FIG. 1) that travels across the drug-loaded insert 8194 in the inlet channel 8140 may cause the controlled-release material 160 to degrade and release the secondary drug 104 (FIG. 1). The microchannel 8192 in the inlet channel 8140 may increase the time of exposure between the primary drug 102 and the drug-loaded insert 8194, thereby increasing the concentration of the secondary drug 104 in the primary drug 102. The primary drug 102 and the secondary drug 104 may exit the infusion head 8130 together through the same cannula 8152, which minimizes the number of needle sticks to the patient.

### 12. Parallel Drug-Containing Reservoir

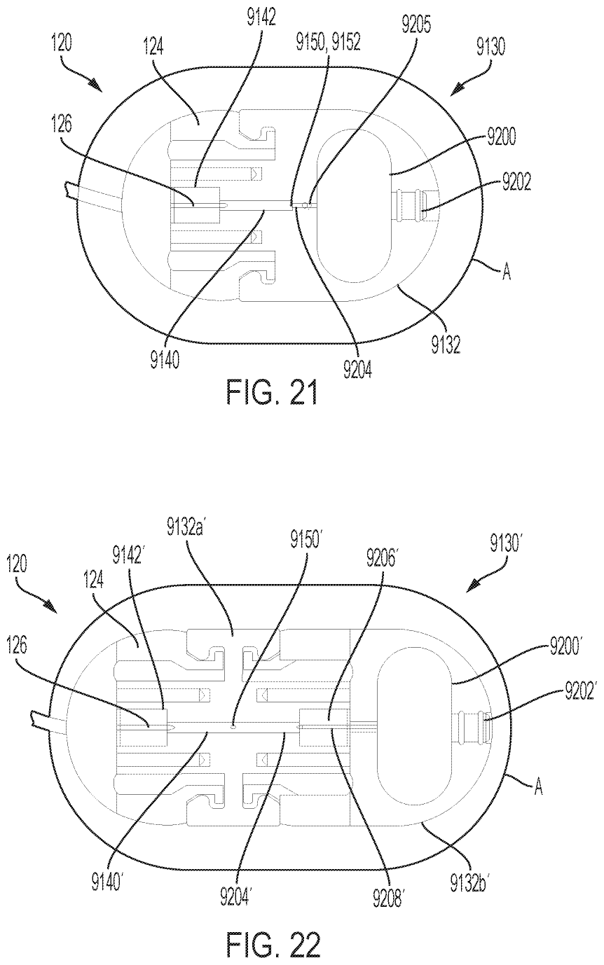

Referring next to FIG. 21, another infusion head 9130 is provided for use with the drug delivery system 100 of FIG. 2. The infusion head 9130 may be similar to the above-described infusion heads 130, 1130, 2130, 3130, 4130, 5130, 6130, 7130, 8130 and their variants, with like reference numerals identifying like elements, except as described below.

The infusion head 9130 includes a housing 9132, an inlet channel 9140 sealed by a septum port 9142, and an outlet channel 9150. The infusion head 9130 also includes a reservoir 9200 that holds the secondary drug 104 (FIG. 1), a fill port containing a stopper 9202 or another suitable seal that seals the reservoir 9200, and a secondary channel 9204 that connects the reservoir 9200 to the outlet channel 9150. The inlet channel 9140 receives the primary drug 102 (FIG. 1) from the needle port 126 of the tubing set 120 through the septum port 9142 and directs the primary drug 102 to the outlet channel 9150, and the secondary channel 9204 directs the secondary drug 104 to the same outlet channel 9150. In this embodiment, the secondary drug 104 may be present in liquid (i.e., injectable) form in the reservoir 9200. The reservoir 9200 may be sized to hold a small volume of the secondary drug 104, such as about 50 microliters to about 500 microliters.

The infusion head 9130 may be designed to limit delivery of the secondary drug 104 relative to the primary drug 102 and/or to inhibit undesired back-flow of the primary drug 102 into the reservoir 9200. In one example, and as shown in FIG. 21, the secondary channel 9204 may be smaller in diameter than the inlet channel 9140. In another example, and as shown in FIG. 21, a check valve 9205 may be provided along the secondary channel 9204. The secondary channel 9204 may remain open until back-pressure (e.g., tissue back-pressure) exceeds a predetermined cracking pressure (e.g., 2 psi) and closes the check valve 9205, thereby preventing delivery of the secondary drug 104 until the back-pressure drops below the cracking pressure.

The infusion head 9130 may be filled by inserting the secondary drug 104 into the housing 9132. More specifically, the infusion head 9130 may be pre-filled by the manufacturer by inserting the secondary drug 104 into the reservoir 9200 and then closing the fill port with the stopper 9202. Alternatively, the infusion head 9130 may be filled by the healthcare provider and/or the patient. In this embodiment, the fill port may contain a septum port (not shown) in place of the stopper 9202, and the infusion head 9130 may be filled by obtaining a syringe with the secondary drug 104 and injecting the secondary drug 104 into the reservoir 9200 through the septum port.

In use, the primary drug 102 and the secondary drug 104 (FIG. 1) travel along parallel flow paths, with the primary drug 102 entering the outlet channel 9150 from the inlet channel 9140 via the septum port 9142 and the secondary drug 104 entering the outlet channel 9150 from the secondary channel 9204. The primary drug 102 and the secondary drug 104 may combine in the outlet channel 9150 and exit the infusion head 9130 together through the same cannula 9152, which minimizes the number of needle sticks to the patient.

Referring next to FIG. 22, a variation of infusion head 9130′ is shown. The infusion head 9130′ includes a multi-piece housing, specifically a first, female-shaped housing 9132a′ and a second, male-shaped housing 9132b′, an inlet channel 9140′ sealed by a septum port 9142′, an outlet channel 9150′, a reservoir 9200′, a stopper 9202′, and a secondary channel 9204′. The infusion head 9130′ also includes an intermediate septum port 9206′ and an intermediate needle port 9208′. The second housing 9132b′ is removably coupled to the first housing 9132a′. As shown in FIG. 22, the first housing 9132a′ carries the inlet channel 9140′, the outlet channel 9150′, and the intermediate septum port 9206′, while the second housing 9132b′ carries the reservoir 9200′, the stopper 9202′, the secondary channel 9204′, and the intermediate needle port 9208′. When the second housing 9132b′ is coupled (e.g., clipped) to the first housing 9132a′, the intermediate needle port 9208′ of the second housing 9132b′ punctures the intermediate septum port 9206′ of the first housing 9132a′ to place the reservoir 9200′ and the secondary channel 9204′ in fluid communication with the outlet channel 9150′. The ability to remove the second housing 9132b′ and its reservoir 9200′ may make it easier to manufacture, fill, and optionally re-fill the reservoir 9200′, even without the user having to couple and/or uncouple the housings 9132a′, 9132b′.

### 13. Independent Drug-Containing Reservoir

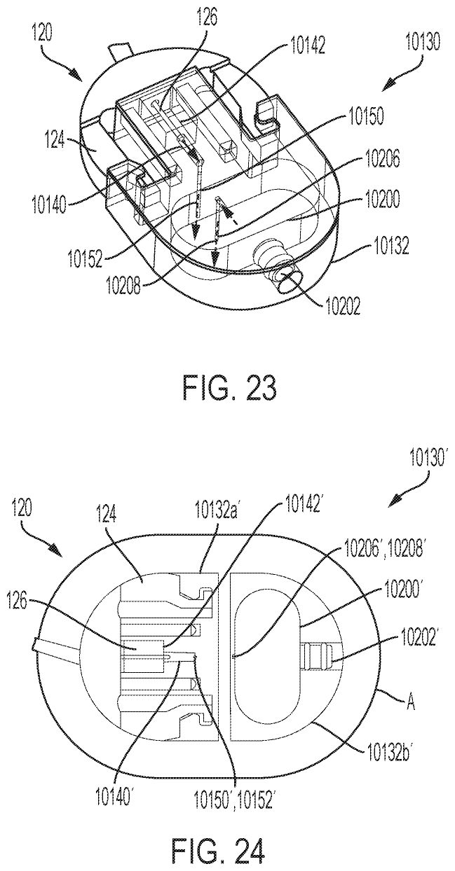

Referring next to FIG. 23, another infusion head 10130 is provided for use with the drug delivery system 100 of FIG. 2. The infusion head 10130 may be similar to the above-described infusion heads 130, 1130, 2130, 3130, 4130, 5130, 6130, 7130, 8130, 9130 and their variants, with like reference numerals identifying like elements, except as described below.

The infusion head 10130 includes a housing 10132, an inlet channel 10140 sealed by a septum port 10142, and an outlet channel 10150 for the primary drug 102 (FIG. 1). The infusion head 10130 also includes a reservoir 10200 that holds the secondary drug 104 (FIG. 1), a stopper 10202 that seals the reservoir 10200, and a secondary outlet channel 10206 for the secondary drug 104 that is independent of the outlet channel 10150 for the primary drug 102. In the illustrated embodiment of FIG. 23, the secondary outlet channel 10206 includes a secondary drug infusion element in the form of a secondary cannula 10208 or a secondary needle. In this embodiment, the secondary drug 104 may be present in liquid (i.e., injectable) form in the reservoir 10200. The reservoir 10200 may be sized to hold a small volume of the secondary drug 104, such as about 50 microliters to about 500 microliters.

The infusion head 10130 may be filled by inserting the secondary drug 104 into the housing 10132. More specifically, the infusion head 10130 may be pre-filled by the manufacturer by inserting the secondary drug 104 into the reservoir 10200 and then closing the fill port with the stopper 10202. Alternatively, the infusion head 10130 may be filled by the healthcare provider and/or the patient. In this embodiment, the fill port may contain a septum port (not shown) in place of the stopper 10202, and the infusion head 10130 may be filled by obtaining a syringe with the secondary drug 104 and injecting the secondary drug 104 into the reservoir 10200 through the septum port.

In use, the primary drug 102 and the secondary drug 104 (FIG. 1) travel along independent flow paths, with the primary drug 102 entering the outlet channel 10150 from the inlet channel 10140 via the septum port 10142 and the secondary drug 104 entering the secondary outlet channel 10206 from the reservoir 10200. The primary drug 102 and the secondary drug 104 may exit the infusion head 10130 independently through separate cannulas 10152, 10208, respectively. The cannulas 10152, 10208 may be positioned in close proximity to each other to strike the same nerves, thereby minimizing patient discomfort.

Referring next to FIG. 24, a variation of infusion head 10130′ is shown. The infusion head 10130′ includes two independent housings, specifically a first, female-shaped housing 10132a′ and a second housing 10132b′. The housings 10132a′, 10132b′ may be coupled to the same adhesive pad A for convenience. As shown in FIG. 24, the first housing 10132a′ communicates with the needle port 126 of the tubing set 120 and carries an inlet channel 10140′ sealed by a septum port 10142′, an outlet channel 10150′, and a cannula 10152′ for the primary drug 102 (FIG. 1), while the second housing 10132b′ carries a reservoir 10200′, a stopper 10202′, a secondary outlet channel 10206′, and a secondary cannula 10208′ for the secondary drug 104 (FIG. 1). The cannulas 10152′, 10208′ may be positioned in close proximity to each other on the adhesive pad A to encourage striking the same nerves, thereby minimizing patient discomfort. Also, because the housings 10132a′ and 10132b′ are not rigidly connected to each other, the cannula 10152′ on the first housing 10132a′ may move slightly relative to the secondary cannula 10208′ on the second housing 10132b′, which may minimize patient discomfort when one or the other cannulas 10152′, 10208′ shifts unexpectedly.

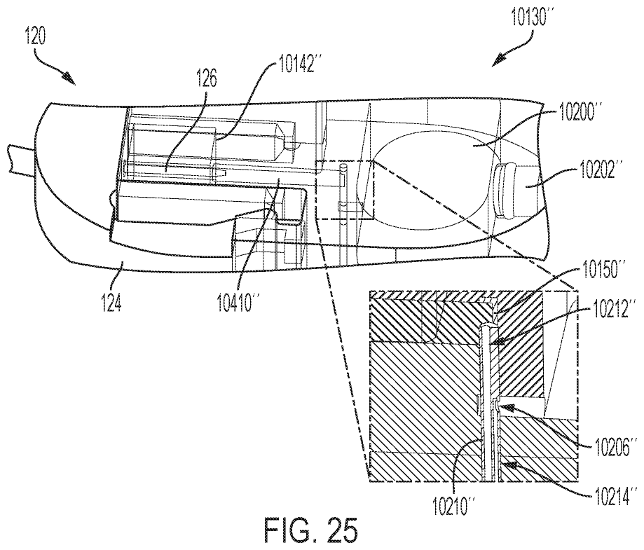

Referring next to FIG. 25, another variation of infusion head 10130 is shown. The infusion head 10130″ includes an inlet channel 10140″ sealed by a septum port 10142″, and an outlet channel 10150″ for the primary drug 102 (FIG. 1) and a reservoir 10200″, a stopper 10202″, and a secondary outlet channel 10206″ for the secondary drug 104 (FIG. 1). The infusion head 10130″ also includes a dual-lumen drug infusion element in the form of a dual-lumen cannula 10210″ or a dual-lumen needle having a primary lumen 10212″ that communicates with the outlet channel 10150″ and a secondary lumen 10214″ that communicates with the secondary outlet channel 10206″. As shown in FIG. 25, the primary lumen 10212″ is circular-shaped and centrally located in the dual-lumen cannula 10210″ and communicates lengthwise with the outlet channel 10150″, while the secondary lumen 10214″ is crescent-shaped, partially surrounds the primary lumen 10212″, and communicates sideways with the secondary outlet channel 10206″. This particular geometric arrangement of the lumens 10212″, 10214″ may provide the dual-lumen cannula 10210″ with a comfortable shape and diameter (e.g., 27 gauge), but this geometric arrangement may vary. In this embodiment, the primary drug 102 and the secondary drug 104 exit the infusion head 10130″ independently through separate lumens 10212″, 10214″, but through the same cannula 10210″, which minimizes the number of needle sticks to the patient.

### 14. Drive Mechanisms for Drug-Containing Reservoirs

Various drive mechanisms may be used to deliver the secondary drug 104 (FIG. 1) from a reservoir. The following drive mechanisms will be shown and described with respect to the reservoir 9200 of the above-described infusion head 9130, but these drive mechanisms may also be used with the above-described infusion heads 9130′, 10130, 10130′, 10130″ or other infusion heads, for example.

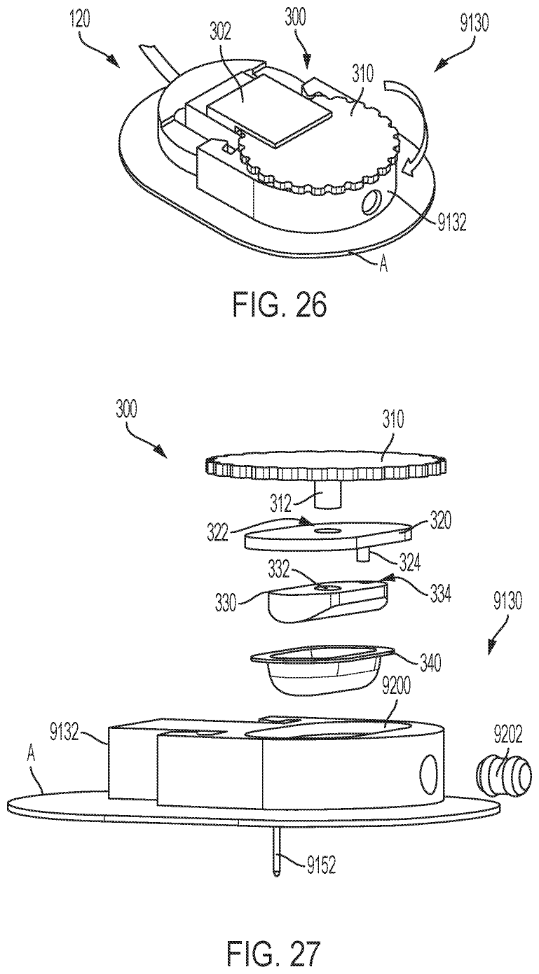

Referring next to FIGS. 26 and 27, the infusion head 9130 includes a manual drive mechanism 300. The manual drive mechanism 300 includes a retention arm 302 coupled to the housing 9132, a manual actuator in the form of a rotatable knob 310, a lid 320, a plunger 330, and a flexible membrane 340, each of which is described further below.

The knob 310 is captured beneath the retention arm 302 (FIG. 26) and includes a threaded post 312 that extends downward into the plunger 330. The knob 310 may be knurled to enhance a user's grip when rotating the knob 310. Although the illustrative drive mechanism 300 includes the rotatable knob 310, other manual actuators such as push buttons, levers, and other actuators are also contemplated herein.

The lid 320 may be coupled (e.g., laser welded, ultrasonically welded, thermally bonded) to the housing 9132 in a sealed manner to capture the plunger 330 and the membrane 340 in the reservoir 9200. The lid 320 includes a central aperture 322 that accommodates the threaded post 312 of the knob 310. The lid 320 also includes an anti-rotation post 324 that extends downward into the plunger 330.

The plunger 330 is captured beneath the lid 320 and acts upon the membrane 340. The plunger 330 includes a threaded nut 332 that engages the threaded post 312 of the knob 310. The plunger 330 also includes an anti-rotation hole 334 that receives the anti-rotation post 324 from the lid 320.

The flexible membrane 340 is captured beneath the lid 320 and the plunger 330 and cooperates with the housing 9132 of the infusion head 9130 to define the top of the reservoir 9200. The flexible membrane 340 may be made from an elastomer, a thin film of thermoplastic, or another flexible material.

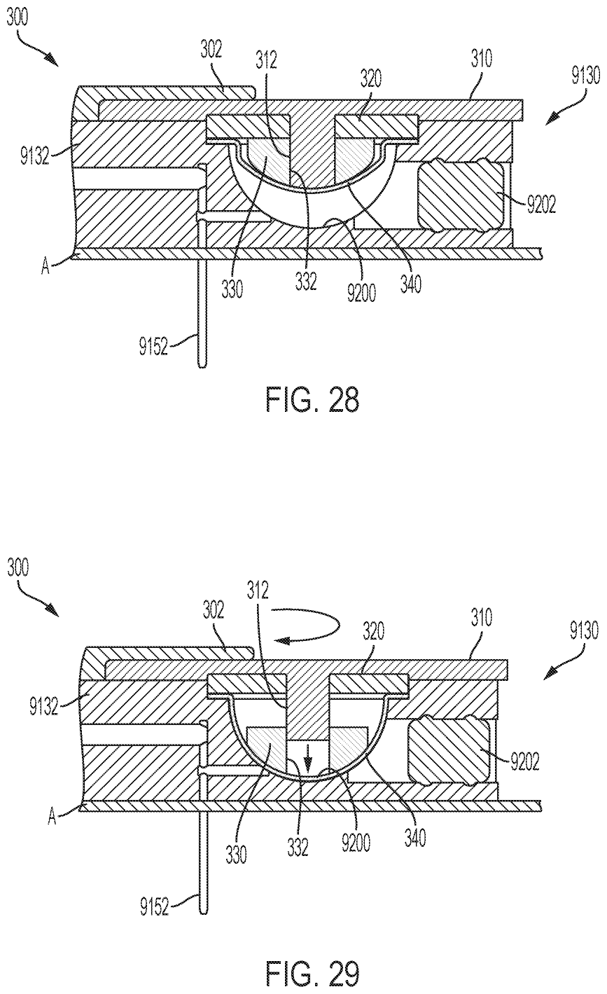

The infusion head 9130 is shown in a ready state in FIG. 28. The membrane 340 is raised to accommodate the secondary drug 104 (FIG. 1) in the reservoir 9200. The reservoir 9200 may be filled in the same manner as described above, such as by injecting the secondary drug 104 through the stopper 9202 and beneath the membrane 340.

The infusion head 9130 is shown in a driven state in FIG. 29. The user rotates the knob 310 by a desired amount beneath the retention arm 302. The rotation of the threaded post 312 of the knob 310 transfers to threaded nut 332 of the plunger 330. Because the anti-rotation post 324 of the lid 320 (FIG. 26) prevents rotation of the plunger 330, the plunger 330 translates downward across the rotating post 312 and against the membrane 340. This downward movement of the membrane 340 expels the secondary drug 104 (FIG. 1) from the reservoir 9200.

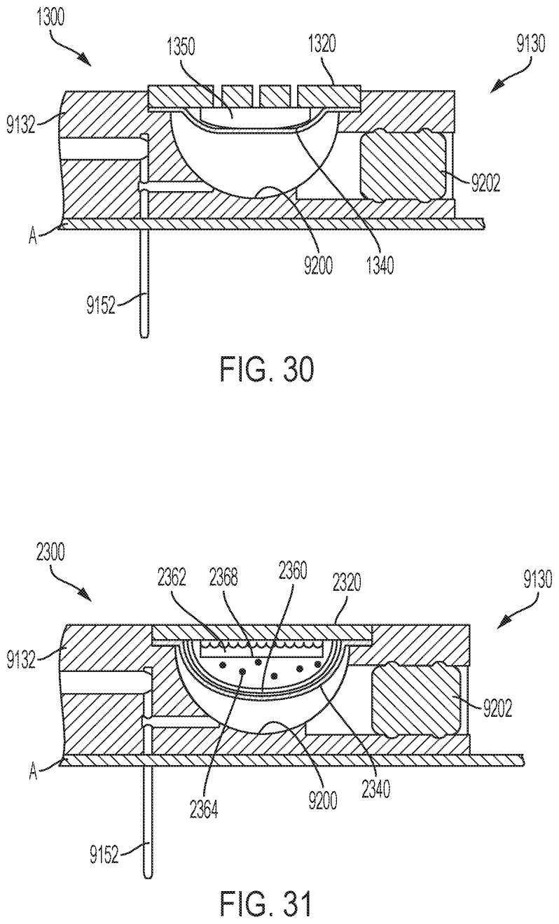

Referring next to FIG. 30, the infusion head 9130 includes a morphic drive mechanism 1300. The morphic drive mechanism 1300 may be similar to the above-described manual drive mechanism 300, with like reference numerals identifying like elements, except as described below.

The morphic drive mechanism 1300 of FIG. 30 includes a perforated lid 1320 and a membrane 1340 that helps define the reservoir 9200. The morphic drive mechanism 1300 also includes a hydromorphic material 1350, such as a hydrogel, that acts upon the membrane 1340. In certain embodiments, the hydromorphic material 1350 may be contained within a second membrane (not shown) that is separate from the membrane 1340 to further separate and minimize exposure between the hydromorphic material 1350 above the membrane 1340 and the secondary drug 104 (FIG. 1) beneath the membrane 1340.

In use, the hydromorphic material 1350 expands as it is exposed to moisture through the perforated lid 1320. This moisture exposure may occur by manually dropping desired amounts of water or other fluid through the perforated lid 1320 or simply by exposure to atmospheric moisture. As the hydromorphic material 1350 expands and presses against the membrane 1340, the membrane 1340 expels the secondary drug 104 (FIG. 1) from the reservoir 9200.

Referring next to FIG. 31, the infusion head 9130 includes a chemical drive mechanism 2300. The chemical drive mechanism 2300 may be similar to the above-described manual drive mechanism 300 and/or morphic drive mechanism 1300, with like reference numerals identifying like elements, except as described below.

The chemical drive mechanism 2300 of FIG. 31 includes a lid 2320 and a membrane 2340 that helps define the reservoir 9200. Above the membrane 2340, the chemical drive mechanism 2300 also includes a first reactant 2362 and a second reactant 2364. The chemical drive mechanism 2300 may further include an optional second membrane 2360 that further separates and minimizes exposure between the reactants 2362, 2364 above the membrane 2340 and the secondary drug 104 (FIG. 1) beneath the membrane 2340.

The reactants 2362, 2364 are configured to chemically react and generate a gas. In one embodiment, the first reactant 2362 is citric acid, and the second reactant 2364 is a bicarbonate powder, which react to generate carbon dioxide gas. Other suitable reactants are provided in U.S. Pat. No. 9,795,740 and U.S. Patent Application Publication No. 2020/0030537, the disclosures of which are incorporated by reference herein in their entireties.

In use, the reactants 2362, 2364 are exposed to one another. This exposure may involve puncturing a seal or a container 2368 that once separated the reactants 2362, 2364. Other mechanisms for exposing the reactants 2362, 2364 to one another are disclosed in the above-incorporated U.S. Pat. No. 9,795,740 and U.S. Patent Application Publication No. 2020/0030537. The gas generated by the reactants 2362, 2364 presses against the membrane 2340, and the membrane 2340 expels the secondary drug 104 (FIG. 1) from the reservoir 9200.

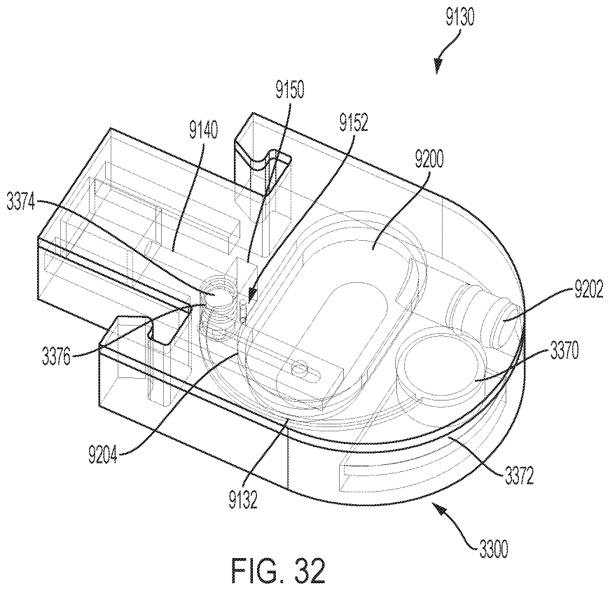

Referring next to FIG. 32, the infusion head 9130 includes an active valve drive mechanism 3300. The active valve drive mechanism 3300 may be similar to the above-described manual drive mechanism 300, morphic drive mechanism 1300, and/or chemical drive mechanism 2300, with like reference numerals identifying like elements, except as described below.

The active valve drive mechanism 3300 of FIG. 32 includes a pressure source, such as a spring-biased piston (not shown), that acts upon and pressurizes the secondary drug 104 (FIG. 1) in the reservoir 9200. The active valve drive mechanism 3300 also includes a battery 3370 or another suitable power source, an electronic control module 3372 powered by the battery 3370, and an electronic piston valve 3374 operated by the electronic control module 3372. As shown in FIG. 32, the electronic piston valve 3374 is located in the secondary channel 9204 between the reservoir 9200 and the outlet channel 9150. The electronic piston valve 3374 is biased closed with a spring 3376.

In use, the electronic control module 3372 sends a signal to open the electronic piston valve 3374. The pressurized secondary drug 104 (FIG. 1) is then able to flow through the open secondary channel 9204 to the outlet channel 9150 for delivery to the patient.

While this invention has been described as having exemplary designs, the present invention can be further modified within the spirit and scope of this disclosure. In one example, the in-line drug-loaded components disclosed with respect to infusion heads 130, 1130, 2130, 3130, 4130, 5130, 6130, 7130, 8103, and their variants, may be used in combination with each other. In another example, the in-line drug-loaded components disclosed with respect to infusion heads 130, 1130, 2130, 3130, 4130, 5130, 6130, 7130, 8103, and their variants, may be used in combination with the drug-containing reservoirs disclosed with respect to infusion heads 9130, 10310, and their variants, with the drug-loaded components delivering a first type of secondary drug and the drug-containing reservoirs delivering a second type of secondary drug. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. An infusion head of a drug infusion system, the drug infusion system including a drug delivery device configured to deliver a primary drug to the infusion head, the infusion head comprising:
a housing including an inlet channel and an outlet channel that cooperate to form a fluid flow path for the primary drug through the housing;
an inlet port coupled to the inlet channel of the housing;
an infusion element coupled to the outlet channel of the housing and configured for insertion in a patient's skin; and
at least one drug-loaded component inserted into the inlet channel or the outlet channel of the housing, the at least one drug-loaded component including a controlled-release material with a secondary drug on at least a surface wetted by the primary drug such that the drug-loaded component releases the secondary drug into the primary drug traveling through the fluid flow path.

2. The infusion head of claim 1, wherein the at least one drug-loaded component is a bushing that supports the infusion element in the outlet channel of the housing, wherein at least an upper surface of the bushing includes the controlled-release material.

3. The infusion head of claim 1, wherein the at least one drug-loaded component is a tube inserted into the inlet channel of the housing, wherein at least an inner surface of the tube includes the controlled-release material.

4. The infusion head of claim 1, wherein the at least one drug-loaded component is a rod inserted into the inlet channel of the housing, wherein at least an outer surface of the rod includes the controlled-release material.

5. The infusion head of claim 4, further comprising a plurality of additional rods arranged side-by-side or end-to-end with the rod of claim 4.

6. The infusion head of claim 1, wherein the at least one drug-loaded component is a plurality of pellets inserted into the inlet channel of the housing, wherein at least an outer surface of each pellet includes the controlled-release material.

7. The infusion head of claim 1, wherein the at least one drug-loaded component is a tunnel inserted into the inlet channel of the housing, wherein at least an inner surface of the tunnel includes the controlled-release material.

8. The infusion head of claim 1, wherein the at least one drug-loaded component is a plate inserted into the inlet channel of the housing, wherein at least a lower surface of the plate includes the controlled-release material.

9. The infusion head of claim 1, wherein the housing includes at least one microchannel within the inlet channel that lengthens the fluid flow path through the housing and increases exposure of the primary drug to the at least one drug-loaded component.

10. The infusion head of claim 1, wherein the housing is a single-piece housing, and the at least one drug-loaded component is sized for insertion into the single-piece housing through the inlet channel or the outlet channel.

11. The infusion head of claim 1, wherein the housing is a multi-piece housing including a first housing and a second housing, and the at least one drug-loaded component is sized for insertion between the first and second housings.

12. The infusion head of claim 1, further comprising a funnel sandwiched between the inlet port and the at least one drug-loaded component in the inlet channel.

13. The infusion head of claim 1, wherein the at least one drug-loaded component is molded from the controlled-release material.

14. The infusion head of claim 1, wherein the at least one drug-loaded component is coated with the controlled-release material.

15. An infusion head of a drug infusion system, the drug infusion system including a drug delivery device configured to deliver a primary drug to the infusion head, the infusion head comprising:
a housing including an upper surface that faces away from a patient's skin, a lower surface that faces toward the patient's skin, and a side surface that spans between the upper surface and the lower surface;
an inlet channel configured to receive the primary drug from the drug delivery device, the inlet channel having a side opening in the side surface of the housing;
an outlet channel configured to receive the primary drug from the inlet channel, the outlet channel having an optional upper opening in the upper surface of the housing and a lower opening in the lower surface of the housing;
an inlet port positioned in the side opening of the inlet channel;
an optional introducer septum positioned in the optional upper opening of the outlet channel;
an infusion element positioned in the lower opening of the outlet channel and configured for insertion in the patient's skin; and
at least one drug-loaded component sized for insertion through the side opening of the inlet channel or the optional upper opening of the outlet channel, the at least one drug-loaded component including a controlled-release material that releases a secondary drug into the primary drug traveling through the housing.

16. The infusion head of claim 15, wherein the at least one drug-loaded component is a bushing sized for insertion through the upper opening of the outlet channel and configured to support the infusion element in the lower opening of the outlet channel.

17. The infusion head of claim 15, wherein the at least one drug-loaded component is a tube, a plurality of rods, or a plurality of pellets sized for insertion through the side opening of the inlet channel.

18. An infusion head of a drug infusion system, the drug infusion system including a drug delivery device configured to deliver a primary drug to the infusion head, the infusion head comprising:
a first housing;
a second housing coupled to the first housing;
a fluid flow path for the primary drug between the first and second housings, the fluid flow path including:
an inlet channel;
at least one microchannel in the inlet channel that lengthens the fluid flow path through the first and second housings; and
an outlet channel in fluid communication with the inlet channel; and
at least one drug-loaded component inserted between the first and second housings in fluid communication with the fluid flow path, the at least one drug-loaded component including a controlled-release material that releases a secondary drug into the primary drug traveling through the fluid flow path.

19. The infusion head of claim 18, wherein the at least one drug-loaded component is a tunnel, a plate, a plurality of rods, a plurality of pellets, or a non-linear insert.

20. A drug infusion system comprising:
a drug delivery device configured to deliver a primary drug;
a tubing set configured to couple to the drug delivery device; and
an infusion head configured to couple to the tubing set, the infusion head comprising:
a housing including an inlet channel and an outlet channel that cooperate to form a fluid flow path for the primary drug through the housing;
an inlet port coupled to the inlet channel of the housing;
an infusion element coupled to the outlet channel of the housing and configured for insertion in a patient's skin; and
at least one drug-loaded component inserted into the inlet channel or the outlet channel of the housing, the at least one drug-loaded component including a controlled-release material with a secondary drug on at least a surface wetted by the primary drug such that the drug-loaded component releases the secondary drug into the primary drug traveling through the fluid flow path.

21. The drug infusion system of claim 20, wherein the primary drug is insulin and the secondary drug is a nonsteroidal anti-inflammatory drug.

22. The infusion head of claim 1, wherein the primary drug is insulin and the secondary drug is a nonsteroidal anti-inflammatory drug.
